(19) United States
(12) Patent Application Publication
(10) Pub. No.: US 2008/0103334 A1
(43) Pub. Date: May 1, 2008

(54) Process For Synthesis Of Gabapentin

(75) Inventors: Ashok Kumar, Mumbai (IN); Satish Rajanikant Soudagar, Mumbai (IN); Avinash Manohar Nijasure, Mumbai (IN); Nalinakshya Balaram Panda, Mumbai (IN); Prashant Gautam, Mumbai (IN); Gajendrasingh Ramsingh Thakur, Mumbai (IN)

Correspondence Address: PROTEUS PATENT PRACTICE LLC, P.O. BOX 1867, NEW HAVEN, CT, 06508, US

(73) Assignee: IPCA Laboratories Ltd, Mumbai (IN)

(21) Appl. No.: 11/923,352

(22) Filed: Oct. 24, 2007

(30) Foreign Application Priority Data
Oct. 26, 2006 (IN) 1762/MUM/2006

(51) Int. Cl. C07C 61/08

(52) U.S. Cl. 562/507

(57) ABSTRACT

A process for preparation of gabapentin comprising a step of obtaining 1,1-cyclohexane diacetic acid monoamide from 1,1-cyclohexane diacetic acid anhydride, wherein said reaction is characterized by the use of ammonia precursor or pre-generated ammonia-isopropanol solution. The invention further discloses preparation of gabapentin and isolation of gabapentin in polymorphic Form II with high yield and purity.

### TECHNICAL FIELD OF INVENTION

The present invention relates to an improved method of preparation of gabapentin, a known pharmaceutical drug, starting from the intermediate 1,1-cyclohexane diacetic acid anhydride via ‘Hofmann’ rearrangement. More specifically, the invention relates to a new process for the preparation of 1,1-cyclohexane diacetic acid monoamide, intermediate useful in the manufacture of gabapentin or its salts.

### BACKGROUND OF THE INVENTION

Gabapentin which is chemically known as 1-aminomethyl-1-cyclohexaneacetic acid is a very well known pharmaceutical drug useful for the treatment of epilepsy and other cerebral disorders. The structure of gabapentin is shown in the Formula I.

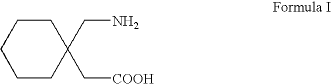

This compound, its process for preparation and use are first disclosed in U.S. Pat. Nos. 4,024,175 and 4,087,544 respectively. The processes disclosed in these patents for gabapentin like derivatives are outlined in the following general scheme which falls in the known methods used for the preparation of primary amines, for example by 1) Curtius rearrangement of cycloalkane diacetic acid monoesters, 2) ‘Hofmann’ rearrangement of cycloalkane diacetic acid monoamides or 3) Lossen rearrangement (Scheme I). The isolation of pure gabapentin is reported to be based on the ion exchange column separation from gabapentin hydrochloride salt.

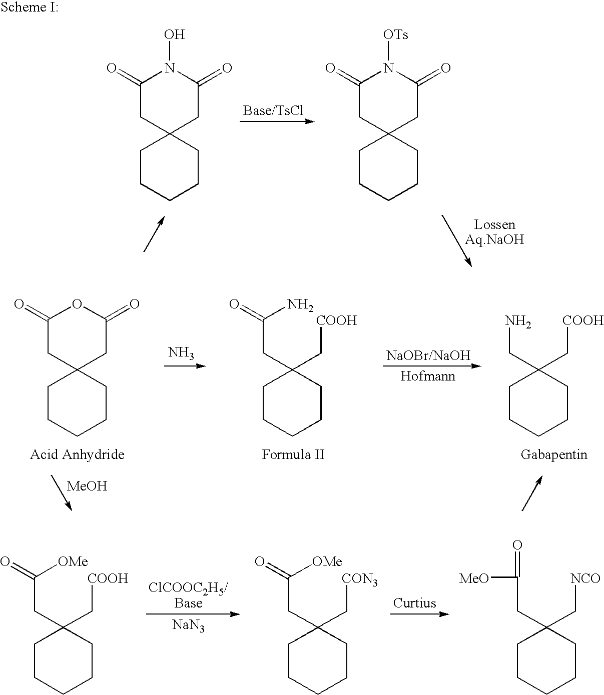

A specific method for gabapentin synthesis given in U.S. Pat. Nos. 4,024,175 and 4,087,544 is as follows (see Scheme I): monomethyl ester of cyclohexane-1,1-diacetic acid was transformed to the corresponding azide which was decomposed (Curtius reaction) in boiling toluene. The resultant isocyanate was hydrolyzed with aqueous hydrochloric acid. The resultant solution was evaporated to dryness to give 1-aminomethylcyclohexane-1-acetic acid hydrochloride, which was converted to gabapentin with a basic ion-exchange resin.

The U.S. Pat. No. 4,024,175 describes the synthesis of the lower cyclic homologous derivative of gabapentin, in particular 1-(methylamino)-1-cyclopentaneacetic acid, through the preparation of cyclopentane diacetic acid monoamide (Formula II in Scheme I), carried out by reaction of the corresponding anhydride with an aqueous ammonia solution. Then ‘Hofmann’ rearrangement of the obtained monoamide, followed by acidification and the extraction of the obtained gabapentin hydrochloride followed by a final purification step consisting in the elution through a basic ion exchange resin followed by recrystallization from alcohols.

In the patent CN 1297885, the preparation of 1,1-cyclohexyl diacetic acid monoamide is described through the reaction of the corresponding anhydride with aqueous or gaseous ammonia in the presence of an organic solvent.

Another report, the International patent application WO 03/002517, having familiar with the above synthetic techniques, describes a process for the synthesis of 1,1-cyclohexane diacetic acid monoamide comprising: a) amination of the anhydride of 1,1-cyclohexane diacetic acid with aqueous ammonia. b) neutralization of the reaction mixture, whereby crude 1,1-cyclohexane diacetic acid monoamide is precipitated and filtered. c) the purification of crude 1,1-cyclohexane diacetic acid monoamide through a crystallization from solvent.

Yet another report, WO2005044779, claims to improve the process for 1,1-cyclohexane diacetic acid monoamide through amination of 1,1-cyclohexane diacetic acid anhydride using aqueous ammonia characterized by temperature below 30° C. and molar amounts of ammonia lower than 3 moles relative to anhydride compound.

Although the processes mentioned above can be considered as an improvement of the process described in the U.S. Pat. No. 4,024,175, but these processes are of not much value when applied in industry, since both methods attempt to use aqueous ammonia solution which leads to partial decomposition of anhydride and lower the yield and purity. Though Chinese patent (CN1297885) describes ammonia gas with organic solvents selected from toluene, benzene etc. as alternative to aqueous ammonia, but the process suffered from its incapability to large scale production due to lack of effective mixing of reactants and reagents since ammonia gas has less solubility in these solvents. The poor solubility of ammonia in the solvents employed in CN 1297885 contributes to low step yield in the synthesis of 1,1-cyclohexane diacetic acid monoamide.

In particular, these methods utilize a considerable amount of reagents and solvents. For example, amination requires considerable amount of ammonia solution which is toxic and has to be disposed. This constitutes extra costs and disposal time. Furthermore, in aqueous reaction system the pH of the solution was required to keep at a specific range to get the reaction to maximum conversion levels and assuring such homogeneity is difficult in industrial level.

Consequently, there is a need for enhanced methodologies for the synthesis of gabapentin under more favourable conditions from the view point of the industrial application of the process, product quality and purity. The present invention addresses this need.

### SUMMARY OF THE PRESENT INVENTION

Thus the object of the present invention is to provide a modification of the known methods for the synthesis of gabapentin which increases yield and minimizes by-products/impurities and which also minimizes the cost of production. A further object is to provide such a method which enables the making of gabapentin in polymorphic form II substantially free from inorganic salts and gabalactam impurity.

These objects and other objects are achieved by studying the process of amidation under varying conditions and surprisingly found improved reaction conditions for the synthesis of 1,1-cyclohexane diacetic acid monoamide, intermediate for the synthesis of gabapentin which ameliorates most of the problems associated with known procedures and is easy to translate to industrial production.

Therefore, the present invention provides a process for 1,1-cyclohexane diacetic acid monoamide, useful in the synthesis of gabapentin and an improved process for gabapentin, wherein the improvement comprises a new process for isolation of Form II polymorphic form of gabapentin. The method is characterized by the use of a precursor of ammonia during the reaction of 1,1-cyclohexane diacetic acid anhydride to obtain 1,1-cyclohexane diacetic acid monoamide. The 1,1-cyclohexane diacetic acid monoamide obtained in this manner is used for the formation of gabapentin.

Thus, according to one embodiment of the present invention, there is provided an improved process for preparation of gabapentin which comprises the steps of:
a) providing a solution of 1,1-cyclohexane diacetic acid anhydride in a solvent;
b) combining it with a precursor which generates ammonia in situ in the reaction solution;
c) effectuating the transformation of 1,1-cyclohexane diacetic acid anhydride into 1,1-cyclohexane diacetic acid monoamide;
d) isolating said 1,1-cyclohexane diacetic acid monoamide from the reaction solution;
e) subjecting the 1,1-cyclohexane diacetic acid monoamide sodium salt in an aqueous solution to a ‘Hofmann’ rearrangement reaction with an aqueous solution of sodium hypobromite (prepared in situ) at a temperature in the range of −5 to −10° C., and further at 55° C., followed by acidification with hydrochloric acid to obtain gabapentin hydrochloride wherein the content of gabalactam is less than 7%;
f) extracting gabapentin hydrochloride from step (e) using ethanol followed by removing ethanol to obtain a residue comprising gabapentin hydrochloride;
g) gabapentin hydrochloride obtained in step (f) is dissolved in a mixture of alcohol and water to form a solution;
h) treating the gabapentin hydrochloride solution of step (g) with triethylamine to form a solution of gabapentin and triethylamine hydrochloride;
i) removing ethanol to give a residue comprising gabapentin and triethylamine hydrochloride
j) treating the mixture from step (i) with a solvent mixture comprising water and acetone to form a solution/slurry;
k) crystallizing gabapentin as Form II from said solution by cooling.

In various embodiments of the above method, the acid may be any equivalent acid such as sulfuric acid, formic acid, acetic acid, and the like. In additional embodiments the ethanol may be substituted by an equivalent alcohol in any step in which ethanol is used.

In a second embodiment, according to the present invention, an improved process for gabapentin is provided which is characterized by preparation of 1,1-cyclohexane diacetic acid monoamide by treatment of 1,1-cyclohexane diacetic acid anhydride in a polar organic solvent such as an alcohol solvent having dissolved ammonia followed by neutralization with acid to recover 1,1-cyclohexane diacetic acid monoamide. 1,1-Cyclohexane diacetic acid monoamide is further reacted to produce gabapentin in the aforementioned manner. The process enables the production of gabapentin with high yield and purity.

### DETAILED DESCRIPTION OF THE INVENTION

While the invention will now be described in detail in connection with certain preferred and optional embodiments, so that various aspects thereof may be more fully understood and appreciated, it is not intended to limit the invention to these particular embodiments.

Thus the present invention provides an improved industrial method for manufacturing gabapentin, more specifically a process for the intermediate 1,1-cyclohexane diacetic acid monoamide for use in the synthesis of gabapentin, wherein the new method ameliorates problems associated with use of aqueous ammonia in 1,1-cyclohexane diacetic acid monoamide formation and isolation of gabapentin substantially free of inorganic salts and gabalactam impurity, and provides reproducibly high yields.

In accordance with the main thrust of the invention, there is provided a new process for preparing 1,1-cyclohexane diacetic acid monoamide intermediate from 1,1-cyclohexane diacetic acid anhydride characterized in that the 1,1-cyclohexane diacetic acid anhydride is contacted with a precursor of ammonia which liberates ammonia as a reactant in situ to react with the anhydride to produce the target monoamide derivative. The precursor which liberates ammonia is selected from ammonium carbonate and a mixture of ammonium chloride with sodium hydroxide. The present inventors have found that these reagents release ammonia in situ within the reaction composition to react with the anhydride derivative to produce the monoamide compound. In an equivalent embodiment the homogeneous ammonia reagent may be provided by a preformed solution of ammonia in a nonaqueous solvent such as an alcohol. The present method has good control on the use of ammonia in the reaction and produces 1,1-cyclohexane diacetic acid monoamide steadily and reproducibly with high yield and purity.

The reaction disclosed herein may be carried out in organic solvent or water or their mixture. It is preferably carried out in an organic solvent which includes, but not limited to, alcohols, ketones, hydrocarbons and chlorinated hydrocarbons. Particularly preferred solvent is toluene, when ammonia precursor is ammonium carbonate. When the precursor is ammonium chloride, the reaction is preferably carried out in presence of water or mixture of water and organic solvent which forms an aqueous single phase. The reaction may be carried at room temperature or with cooling or heating.

In a second embodiment of the present invention, there is provided a second method for preparation of 1,1-cyclohexane diacetic acid monoamide characterized in that the 1,1-cyclohexane diacetic acid anhydride is treated with a pregenerated solution of ammonia in a polarorganic solvent such as an alcohol. The method is simple, and economical; operation conditions are mild and can be scaled to industrial production. Preferably the alcohol used in the ammonia-alcohol solution is isopropyl alcohol, although other alcohols such as n-propyl alcohol, and homologous alcohols, may also be used. It is preferred to use ammonia-alcohol solution having 11 to 14% ammonia in isopropyl alcohol. The reaction may be carried out with appropriate cooling or heating or at ambient temperature, and preferably it is carried out under chilling at about 0 to 10° C. temperature range. After complete conversion of 1,1-cyclohexane diacetic acid anhydride to 1,1-cyclohexane diacetic acid monoamide, it is isolated by neutralization and crystallization from suitable solvent, such as isopropyl alcohol. In case where aqueous solvents are used, the isolation of 1,1-cyclohexane diacetic acid monoamide is carried out using conventional methods such as extractive work-up after neutralization. Neutralization may be carried out in a conventional manner by acidification with an organic or inorganic acid.

The process for conversion of 1,1-cyclohexane diacetic acid monoamide into gabapentin is illustrated as follows.

A solution of 1,1-cyclohexane diacetic acid monoamide in aqueous sodium hydroxide is provided at one place. This solution is added to a hypobromite aqueous solution. Equivalent hypohalites, or similar oxidizing agents, may also be used. Preferably the hypobromite aqueous solution is prepared in situ prior to the addition of 1,1-cyclohexane diacetic acid monoamide by combining bromine with aqueous sodium hydroxide. Preferably the addition of 1,1-cyclohexane diacetic acid monoamide sodium salt solution to the sodium hypobromite solution is carried out with chilling, preferably at a temperature range of −5 to −10° C. and maintained the reaction solution at this temperature for about 30 minutes and then further maintained at 35-40° C. for 1 hour. The reaction mixture is then acidified with an inorganic acid, or an organic acid, such as hydrochloric acid, to form a gabapentin salt, such as gabapentin hydrochloride. The aqueous solution may be washed with an organic solvent such as a hydrocarbon or a chlorinated hydrocarbon solvent to remove any organic gabalactam impurity if present. The mixture there after is optionally concentrated and the gabapentin hydrochloride is isolated by filtration or extracted with an alcohol, such as ethanol, or any equivalent alcohol. The extraction may be repeated one or more times and the ethanol solution as such used for formation of gabapentin. Alternately ethanol may be evaporated and gabapentin hydrochloride is re-extracted with ethanol for getting better purity.

The aqueous ethanol solution of the gabapentin salt, such as gabapentin hydrochloride, is treated with an organic base such as triethylamine to form a solution of free gabapentin in aqueous ethanol. Ethanol from this solution is evaporated to form a residue comprising gabapentin and hydrochloride salt or similar salt of the organic base. The mixture may be optionally treated with a solvent such as dichloromethane. The mass is then heated to dissolve in acetone-water solvent mixture, cooled and chilled to precipitate pure gabapentin. The polymorphic form of gabapentin isolated shows the characteristics of Form II as given in the U.S. Pat. No. 6,255,526B₁ and XRPD pattern as given in WO2004/093779.

In a second aspect of the invention there is provided an improved method for the manufacture of gabapentin using 1,1-cyclohexane diacetic acid monoamide manufactured in accordance with the present invention wherein the isolation of gabapentin free of chloride ions and contaminate, gabalactam impurity, yet avoiding the use of ion exchange column taught in the prior art. The method comprises the following:
a) subjecting 1,1-cyclohexane diacetic acid monoamide or its sodium salt to a ‘Hofmann’ rearrangement reaction with an aqueous solution of sodium hypobromite (prepared in situ) at a temperature in the range of −5 to −10° C., and further at 55° C., followed by acidification with hydrochloric acid to obtain gabapentin hydrochloride wherein the content of gabalactam is less than 7%;
b) extracting gabapentin hydrochloride from step (a) using ethanol followed by removing ethanol to obtain a residue comprising gabapentin hydrochloride;
c) gabapentin hydrochloride obtained in step (b) is dissolved in a mixture of alcohol and water to form a solution;
d) treating the gabapentin hydrochloride solution of (c) with triethylamine to form a solution of gabapentin and triethylamine hydrochloride;
e) removing ethanol to give a residue comprising gabapentin and triethylamine hydrochloride;
f) treating the mixture from step (e) with a solvent mixture comprising water and acetone to form a solution/slurry;
g) crystallizing gabapentin Form II from said solution by cooling.

In step f), the volume of water is between 1 to 4 and acetone is between 4 to 20, and especially preferred is 1 to 3 volume of water and 4 to 15 volume of acetone, relative to weight of starting gabapentin. The proportion of water to acetone in the mixture is preferably in the range of 1:3 to 1:6, and more preferably in the range of 1:4 to 1:5.

In earlier methods gabapentin is synthesized by forming gabapentin hydrochloride using a suitable method, and then subjecting the gabapentin hydrochloride salt to ion exchange treatment. This process provides free gabapentin in a very dilute aqueous solution, and during the extraction process to isolate the gabapentin lactamisation occurs, leading to contamination. Water being difficult to distill, and the resulting aqueous solution having very low concentration of gabapentin, the process of evaporation is a very tedious and time and energy consuming operation. Such processes are not cost-efficient and not user friendly and therefore may not be suitable for industrial applications.

On the contrary the improved process of the present invention employs a crystallization process to isolate the pure gabapentin substantially free of both chloride ions and gabalactam.

The gabapentin obtained by the process of the present invention, may be formulated into a dosage form, e.g., tablet, capsule, etc., by combining with one or more pharmaceutically acceptable excipients using known techniques. The resulting dosage form may include a suitable amount of the active ingredient and other pharmaceutical agents. Further, the dosage form may be immediate release or extended release. The dosage forms prepared by the process of the present invention may be administered to a mammal in need, for treatment of epilepsy and other cerebral disorders.

The following examples, which include preferred embodiments, is intended to illustrate the practice of this invention, it being understood that the particulars shown are by way of example and for purpose of illustrative discussion of preferred embodiments of the invention.

### EXAMPLES

### Example 1

### Preparation of 1,1-Cyclohexanediacetic Acid Monoamide

In 1.0 lit R B Flask charged water (135 ml), and ammonium chloride (161.6 g), under cooling. Aqueous sodium hydroxide solution (110 g in 200 ml water) was added drop wise under stirring maintaining temperature below 20° C. After complete addition the reaction mass was further maintained at 0-5° C. for 30 min. Charged 1,1-cyclohexane diacetic acid anhydride (100 gm) maintaining temperature below 5° C. Maintained the reaction mass for 2.0 hrs at about 0° C., then raised the temperature to 25-30° C. and maintained 2 hrs at room temperature. Adjusted pH of the solution to 2-3 with aqueous HCl. Cooled the reaction mass to 0° C., maintained for 1.0 hr, filtered the solid mass and washed with chilled water. Dried the wet cake to get 105 gm 1,1-Cyclohexanediacetic acid monoamide.

### Example 2

### Preparation of 1,1-Cyclohexanediacetic Acid Monoamide

In 1.0 lit R B Flask charged Toluene (500 ml) and 1,1-cyclohexanediacetic acid anhydride (100 gm). Heated the reaction mixture to 35-40° C. and charged slowly ammonium carbonate (47 g). Raised the temperature gradually to 70° C. and maintained for 6-8 hrs at ˜70° C. Cooled the reaction mass to 0° C. and filtered the same. The solid obtained was taken in water and adjusted the pH to 2-3 with conc. HCl. The solution was cooled to 0° C. and maintained 2-3 hrs, filtered and washed with chilled water. Dried the wet cake to get 107 g 1,1-Cyclohexane diacetic acid monoamide.

### Example 3

### Preparation of 1,1-Cyclohexanediacetic Acid Monoamide

In 1.0 lit R B Flask charged 600 ml 12-14% ammonia solution in isopropyl alcohol and cooled to 0° C. Slowly charged 1,1-cyclohexanediacetic acid anhydride (100 g) maintaining temperature below 5° C. Stirred 1.0 hr after complete addition and raised the temperature to room temperature. Then cooled to 15-20° C. and adjusted the pH to 2-3 with aq. HCl solution. The reaction mass was stirred further for about 15 hrs, Filtered and washed the solid with chilled water. Dried the wet cake to get 95 g 1,1-Cyclohexanediacetic acid monoamide.

The filtrate was further concentrated to recover second crop 7 g.

### Example 4

### Preparation of Gabapentin Hydrochloride (Crude)

In a 1 litre flask, 350 ml water, 98.5 g (2.46 moles) sodium hydroxide flakes were mixed to get a clear solution. Then it was cooled to −8° C., and 28.6 ml (0.554 moles) liquid bromine was added drop-wise over a period of 40 minutes. To this a solution of 1,1-cyclohexanediacetic acid monoamide in aqueous sodium hydroxide solution was added slowly with vigorous stirring. After addition the mixture was maintained at −8 to −5° C. for 1 hour under stirring, then allowed the temperature to rise to room temperature and further maintained at 35-40° C. for 1 hour. The mixture then cooled to 15-20° C. and the pH of the mixture adjusted to 5 and washed with dichloromethane. The pH of the aqueous layer further adjusted to 2 using conc. HCl and stirred for 4 hours. The precipitate obtained was filtered and dried to obtain 95 gm Gabapentin hydrochloride salt (crude).

### Example 5

### Purification of Gabapentin Hydrochloride

The crude gabapentin hydrochloride salt obtained above was taken in 285 ml absolute ethanol and stirred at 25° C. for 2 hours, and then filtered to remove insolubles. Ethanol was distilled from solution to obtain gabapentin hydrochloride (Yield 84 gm).

### Example 6

### Preparation of Gabapentin (Crude)

The gabapentin hydrochloride pure (84 gm) obtained above was added into a mixture of 170 ml ethanol and 45 ml water. The pH of the solution was then adjusted to 7.2 by adding triethyl amine and distilled off the solvent completely. The solid obtained was washed with acetone/water and dried to yield 59.0 gm gabapentin (crude).

### Example 7

### Purification of Gabapentin Crude

The gabapentin crude obtained above (59 g) was added to 177 ml water and stirred until dissolution under heating. The solution was filtered and filtrate was chilled to 0-5° C. To this solution acetone (885 ml) was added and mixture was maintained at 0-5° C. The precipitate was filtered and dried under vacuum to obtain 50.0 gm gabapentin (pure)

We claim:

1. A process for preparation of gabapentin (Formula I), 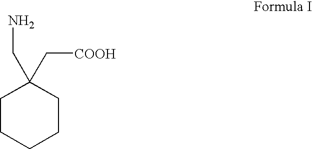
comprising the steps of
a) obtaining 1,1-cyclohexane diacetic acid monoamide (Formula II) 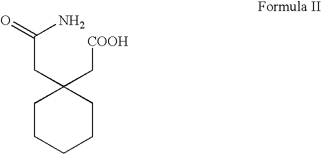
by reacting 1,1-cyclohexane diacetic acid anhydride with a composition that generates free ammonia in solution or that comprises a solution of ammonia in a polar organic solvent,
b) converting 1,1-cyclohexane diacetic acid monoamide to gabapentin, and
c) purifying gabapentin.

2. The process as claimed in claim 1, wherein the composition comprises ammonium carbonate, a mixture of ammonium chloride and sodium hydroxide, or a pre-generated ammonia-isopropanol solution.

3. The process as claimed in claim 1, wherein the reaction is carried out in a solvent.

4. The process as claimed in claim 3, wherein the solvent is water, organic solvent or mixtures thereof.

5. The process as claimed in claim 3, wherein the solvent is an alcohol, a ketone, a hydrocarbon or a chlorinated hydrocarbon.

6. The process as claimed in claim 3, wherein the solvent is toluene, isopropanol or their mixture with water.

7. The process as claimed in claim 1, wherein the 1,1-cyclohexane diacetic acid monoamide is isolated.

8. The process as claimed in claim 7, wherein the isolation comprises neutralization followed by crystallization of 1,1-cyclohexane diacetic acid monoamide from isopropyl alcohol.

9. The process as claimed in claim 1, wherein the 1,1-cyclohexane diacetic acid monoamide or its sodium salt is reacted with an aqueous solution of sodium hypohalite to obtain gabapentin.

10. The process as claimed in claim 9, further wherein said gabapentin is purified by the following steps:
a. acidifying the reaction mass obtained in claim 9 to provide a gabapentin salt, followed by filtration or extraction of the gabapentin salt using an alcohol;
b. evaporating the alcohol, followed by dissolving the residue in a mixture of water and alcohol;
c. adding triethylamine to form a solution of free gabapentin and a triethylamine salt;
d. evaporating the solvent to develop a residue comprising gabapentin and the triethylamine salt;
e. dissolving the residue from step (d) in a second solvent mixture comprising acetone and water; and
f. crystallizing gabapentin from said solution by cooling.

11. The process as claimed in claim 9, wherein the hypohalite is sodium hypobromite.

12. The process as claimed in claim 9, wherein the purified gabapentin is substantially free of gabalactam and inorganic ions.

13. A process for synthesizing gabapentin comprising the steps of:
a. reacting 1,1-cyclohexane diacetic acid monoamide or its sodium salt with an aqueous solution of sodium hypohalite to obtain gabapentin;
b. acidifying the reaction mass obtained in step a), followed by filtration or extraction of a gabapentin salt using an alcohol;
c. evaporating the alcohol to provide a residue comprising the gabapentin salt, followed by forming a solution of the gabapentin salt in a mixture of water and alcohol;
d. adding triethylamine to form a solution of free gabapentin and a triethylamine salt;
e. evaporating the solvent to develop a residue comprising gabapentin and the triethylamine salt;
f. dissolving the residue from step (e) in a second solvent mixture comprising acetone and water; and
g. crystallizing gabapentin from said solution by cooling.

14. The process as claimed in claim 13, wherein the proportion of acetone:water in step (f) is 1:3 to 1:6 and the volume in the range of 1 to 4 by weight of water, and 4 to 20 by weight of acetone, relative to the weight of crude gabapentin.

15. A process for synthesizing gabapentin comprising the following steps:
a. reacting 1,1-cyclohexane diacetic acid anhydride with a composition that generates free ammonia in solution or that comprises a solution of ammonia in a polar organic solvent,
b. to obtain 1,1-cyclohexane diacetic acid monoamide;
c. reacting 1,1-cyclohexane diacetic acid monoamide or its sodium salt with an aqueous solution of sodium hypohalite to obtain gabapentin;
d. acidifying the reaction mass obtained in step b), followed by filtration or extraction of a gabapentin salt using an alcohol;
e. evaporating the alcohol to provide a residue comprising the gabapentin salt, followed by forming solution in a mixture of water and alcohol;
f. adding triethylamine to form a solution of free gabapentin and a triethylamine salt;
g. evaporating the solvent to develop a residue comprising gabapentin and the triethylamine salt;
h. dissolving the residue from step (e) in a second solvent mixture comprising acetone and water; and
i. crystallizing gabapentin from said solution by cooling

16. The process as claimed in claim 16, wherein the composition comprises ammonium carbonate, a mixture of ammonium chloride and sodium hydroxide, or a pre-generated ammonia-isopropanol solution.

17. The process as claimed in claim 16 further comprising a step of forming a pharmaceutical dosage form comprising gabapentin

18. The process as claimed in claim 1 wherein the gabapentin is in Form II.

19. The process as claimed in claim 13 wherein the gabapentin crystallizes in Form II.

20. The process as claimed in claim 15 wherein the gabapentin crystallizes in Form II.
